(19) United States
(12) Patent Application Publication
(10) Pub. No.: US 2007/0166249 A1
(43) Pub. Date: Jul. 19, 2007

(54) Pharmaceutical and cosmetic formulations for treating fingernails

(76) Inventor: Hans Meyer, Riehen (CH)

Correspondence Address: WENDEROTH, LIND & PONACK, L.L.P., 2033 K STREET N. W., SUITE 800, WASHINGTON, DC, 20006-1021, US

(21) Appl. No.: 10/569,664

(22) PCT Filed: Aug. 23, 2004
(86) PCT No.: PCT/CH04/00533

§ 371 Date: Nov. 6, 2006

(30) Foreign Application Priority Data
Aug. 25, 2003 (EP) 030191571

(51) Int. Cl. A61K 31/37; A61K 31/35

(52) U.S. Cl. 424/61

(57) ABSTRACT

Topically applicable agents are described, for the treatment of nail diseases and for nail care, which contain where needed, besides one or more active substance and physiologically compatible active ingredient and solution mediator, one or more compounds of the formula I
R—O—R₁ (I) where R represents a straight or branched alkyl residue with 5-8 carbon atoms and
R₁ represents a formyl group or an acetyl group

The present invention relates to topical application products for the treatment of nail diseases and nail care with improved penetration properties through the nail substance and the skin.

The direct topical treatment of nail diseases and the nail care proceed practically free of side-effects, are very simple to carry out and cause only minimal costs. However, the essential problem of the direct topical use of nail compositions consists in carrying the active substances including nutrient and anabolic substances in sufficient amounts through the nail into the deeper situated tissue layers and into the nail root, completely destroying the pathogens present and providing the nail with nutrient and anabolic substances. With conventional products it is possible to ease the symptoms by direct topical treatment; however, in the regular case they reappear after termination of the treatment.

It has already been proposed to improve the results of the treatment with the direct topical use of active substances in that the active substances were used together with a so-called carrier, i.e. a substance which in addition to a good solubility for the active substance also possesses a good penetrability through the nail substance and the ability to transport the active substance through the nail tissue. As an example, EP-A-0 503 988 describes medicaments for the treatment of onychomycoses, which contain besides an, antimycotic active substance at least partly soluble in water and a C₂-C₈ alcanol, straight or branched, as well as a medium consisting of at least to one third of water, one hydrophilic substance promoting the penetration of the antimycotic through the nail. Penetration promoting substances are e.g. glycol, monoether glycol, diether glycol, dimethylsulfoxide, caprolactams, dimethylisosorbid, isopropylidenglycerin, dimethylimidazolidinone, N-methylpyrolidone-2, pyrolidone-2, ethylacetate, glycerides of C₈-C₁₂ polyoxyethylenes and polyethylenglycol-glyceryllaurate and dimethylacetamide. The formulation principle described in EP-A-0 503 988 is, in view of the partial solubility in water postulated for the active substance, only suitable for a limited number of active substances i.e. unsuitable for many active ingredients.

As yet, no satisfactory product for topical treatment of nail diseases and for topical nail care exists, containing a carrier that allows for the transport of the required amount of active substance through the nail into the nail root(matrix), necessary for a successful treatment

It is therefore the task of the present invention to solve the problems connected with the topical treatment of nail diseases and the topical nail care and to provide pharmaceutical and cosmetic products that offer a successful treatment.

It was found that compounds of the formula (I)
R—O—R₁ (1)
where
R represents a straight or branched alkyl residue with 5-8 carbon atoms and
R₁ represents hydrogen, a formyl group or an acetyl group
not only possess excellent penetration ability through the keratinized nail substance and the bordering skin but can also transport, both therapeutic active substances, and antimycotics, antibiotics, antiseptics and corticosteroids, as well as other nail care substances such as important nutrients through the keratinized nail and through the skin.

Object of the present invention is therefore a topically applicable agent for the treatment of nail diseases and for nail care, containing

(a) one or more therapeutic or nurturing active substance,

(b) one or more compounds of the formula I
R—O—R₁ (I)
where
R represents a straight or branched alkyl residue with 5-8 carbon atoms and
R₁ represents hydrogen, a formyl group or an acetyl group
(c) if necessary physiologically compatible adjuvant.

Subject to the present invention, these compounds of the formula I, for the promotion of penetration include formic acid and acetic acid esters of C₅-C₈ alcanols. The above formula I includes both formates and acetates of straight primary and secondary C₅-C₈ alcanols as well as their branched ones, isomer alcanols. Individual representatives of C₅-C₈ alcanols in the formula I are 1-pentanol (amylalcohol), 3-methyl-1-butanol (Isoamylalcohol), 1-hexanol, 2-hexanol, 4-methyl-1-pentanol, 4-methyl-2-pentanol, 1-heptanol, 2-heptanol, 5-methyl-1-hexanol, 5-methyl-2-hexanol, 1-octanol, 2-octanol, 6-methyl-1-heptanol, 6-methyl-2-heptanol. Of the mentioned primary and secondary C₅-C₈ alcanols the C₅-C₆ alcanols are favoured. Particularly favoured are pentanols, in particular 1-hexanol and 2-hexanol. Mixtures. Still favoured are mixtures of two or several formic and/or ethyl acetates of C₅-C₈ alcanols. Particularly favourable is a mixture of esters of hexanols and heptanols, e.g. 1-hexanol and 1-heptanol, whereby the mixing ratio can vary from 0.5:1.5 to 1.5:0.5. Individual representatives of formates and acetates C₅-C₈ alcanols of the formula I are amyl formiate, amyl acetate, isoamyl formiate, isoamyl acetate, 1-hexyl formiate, 1-hexyl acetate, 2-hexyl formiate, 2-hexyl acetate, 1-heptyl formiate, 1-heptyl acetate, 2-heptyl formiate, 2-heptyl acetate, 1-octyl formiate, 1-octyl acetate, 2-octyl formiate und 2-octyl acetate. Favoured C₅-C₈ alkyl esters are C₅-C₈ alkyl acetates. Particularly favoured are C₅-C₆ alkyl. Still favoured are mixtures of several C₅-C₆ alkyl acetates.

Subject to the present invention for the topically applicable agents basically all therapeutic active substances of synthetic and natural origin come into consideration, which are effective in nail and periungual diseases.

Furthermore nutrients and anabolic substances which are effective in nail care come into consideration as active substances.

Suitable therapeutic active substances, which can be contained in the invented topical agents for the treatment of nail diseases, are antimycotics of synthetic and natural origin, antibiotics, antiseptics and corticosteroids, as well as combinations of the active ingredients mentioned.

Particularly suitable active substances are antimycotics of synthetic and natural origin and nutrients and anabolic substances, which are effective in nail care.

Special examples of therapeutic active substances are:
antimycotics and their physiologically acceptable salts, such as e.g. (±)-cis-2,6-dimethyl-4-[2-methyl-3-(p-tert-pentyl-phenyl)propyl]morpholine (amorolfine), amphotericine, 6-cyclohexyl-1-hydroxy-4-methyl-2(1H)pyridinone (ciclopirox), bis-phenyl-(2-chlorophenyl)-1-imidazolylmethane (clotrimazole), 1-[2-(2,4-dichlorophenyl)-2-(4-chlorobenzyloxy)-ethyl]-imidazole (econazole), 2,4-difluoro-α,α-bis(1H-1,2,4-triazol-1-ylmethyl)benzylalcohol (fluconazole), 5-fluorocytosine (flucytosine), 7-chloro-trimethoxy-methylspiro-[benzofurane-cyclohexene]-dione (griseofulvine), 1-[2,4-dichloro-β-(2,6-dichlorobenzyloxy)-phenethyl]-imidazole (isoconazole), (±)-1-sec-butyl-4-{4-[4-(4-{[(2R*,4S*)-2-(2,4-dichlorophenyl)-2-(1,2,4-triazol-1-ylmethyl)-1,3-dioxolane-4-yl]methoxy}-phenyl)-1-piperazinyl]phenyl}-4,5-dihydro-1,2,4-triazole-5-one (itraconazole), (±)-cis-1-acetyl-4-{4-([2-(2,4-dichlorophenyl)-2-(1H-imidazol-1-ylmethyl)-1,3-dioxolane-4-yl]methoxy)phenyl}piperazine (ketoconazole), 1-[2,4-dichloro-β-(2,4-dichlorobenzyloxyl)-phenethyl]-imidazole(miconazole), (E)-N-cinnamyl-N-methyl-1-naphthylmethylamine (naflifine), nystatine, (E)-N-(6,6-dimethyl-2-heptene-4-ynyl)-N-methyl-1-naphthylmethylamine (terbinafine), 1[2-{(2-chloro-3-thienyl)methoxy}-2-(2,4-dichlorophenyl)ethyl]-1H-imidazole (tioconazole), O-2-naphthyl-N-methyl-N-(3-tolyl)-thiocarbamate (tolnaftate).

Preferred antimycotics according to the present invention are

(±)-cis-2,6-dimethyl-4-[2-methyl-3-(p-tert-pentyl-phenyl)propyl]morpholine (amorolfine), bis-phenyl-(2-chlorphenyl)-1-imidazolylmethane (clotrimazol), 1-[2,4-dichlor-β-(2,6-dichlorbenzyloxy)-phenethyl]-imidazole (Isoconazole), 2,4-difluor-α,α-bis(1H-1,2,4-triazol-1-ylmethyl)benzylalkohol (fluconazole), (±)-1-sec-butyl-4-{4-[4-(4-{[(2R*,4S*)-2-(2,4-dichlorphenyl)-2-(1,2,4-triazole-1-ylmethyl)-1,3-dioxolane-4-yl]methoxy}phenyl)-1-piperazinyl]phenyl}-4,5-dihydro-1,2,4-triazole-5-on (itraconazole), (±)-cis-1-acetyl-4-{4-([2-(2,4-dichlorphenyl)-2-(1H-imidazol-1-ylmethyl)-1,3-dioxolane-4-yl]methoxy)phenyl}piperazine (ketoconazole), 1-[2,4-dichlor-β-(2,4-dichlorbenzyloxyl)-phenethyl]-imidazole (miconazole), (E)-N-(6,6-dimethyl-2-hepten-4-inyl)-N-methyl-1-naphthylmethylamine (terbinafine), α-(2,4-difluorophenyl)-5-fluoro-β-methyl-α-(1H-1,2,4-triazol-1-ylmethyl)-4-pyrimidinethanol (voriconazole).

Particularly preferred antimycotics according to the present invention are

(±)-cis-2,6-dimethyl-4-[2-methyl-3-(p-tert-pentyl-phenyl)propyl]morpholine (amorolfine), bis-phenyl-(2-chlorphenyl)-1-imidazolylmethane (clotrimazole), 1-[2,4-dichlor-β-(2,6-dichlorbenzyloxy)-phenethyl]-imidazole (isoconazole), (±)-1-sec-butyl-4-{4-[4-(4-{[(2R*,4S*)-2-(2,4-dichlorphenyl)-2-(1,2,4-triazole-1-ylmethyl)-1,3-dioxolane-4-yl]methoxy}phenyl)-1-piperazinyl]phenyl}-4,5-dihydro-1,2,4-triazole-5-on (itraconazole), (±)-cis-1-acetyl-4-{4-([2-(2,4-dichlorphenyl)-2-(1H-imidazole-1-ylmethyl)-1,3-dioxolane-4-yl]methoxy)phenyl}piperazine (ketoconazole).
antimycotics of natural origin, such as e.g. etheric oils and plant extracts

Preferred antimycotics of natural origin are tea tree oil (Melaleuca alternifolia), lavender oil (Lavandula officinalis chaix), Australian blue cypress oil (callitis intratropica) and leaf extract of the nim tree (Azadirachta indica). These natural antimycotics can be used as single active substances or as combinations of several such active substances. A preferred combination of active ingredients is a mixture of lavender oil, tea tree oil and Australian blue cypress oil.

antibiotics and their physiologically acceptable salts, such as e.g. α-amino-4-hydroxybenzylpenicillin (amoxicillin), D-(−)-α-aminobenzylpenicillin (ampicillin), 3,3-dimethyl-7-oxo-6-phenylacetamido-4-thia-1-azabicyclo-[3.2.0]-heptane-2-carboxylic acid (benzylpenicillin), benzylpenicillin-benzathine, 3-chloro-7-D-(2-phenylglycinamido)-cephalosporanic acid (cefaclor), 7β-[D-2-amino-(4-hydroxyphenyl)-acetylamino]-3-methyl-cephalosporanic acid (cefadroxil), amino-phenylacetamido-methyl-cephalosporanic acid (cefalexin), D(−)-threo-2-dichloroacetamido-1-(4-nitrophenyl)-1,3-propanediole (chloramphenicole), 1-cyclopropyl-6-fluoro-1,4-dihydro-4-oxo-7-(piperazinyl)-3-quinolinecarboxylic acid (ciprofloxacin), (Z)-(2R,5R)-3-(2-hydroxyethylidene)-7-oxo-4-oxa-1-azabicyclo[3.2.0]heptane-2-carboxylic acid (clavulanic acid), 7-chloro-7-desoxy-lincomycin (clindamycin), 6-desoxy-5-hydroxytetracycline (doxycyclin), 1-ethyl-6-fluoro-1,4-dihydro-4-oxo-7-(1-piperazinyl)-1,8-naphthyridin-3-carboxylic acid (enoxacin), erythromycin, 3-(2-chloro-6-fluorophenyl)-5-methyl-4-isoxazolyl-penicillin (flucloxacillin), kanamycin, lincomycin, 7-dimethylamino-6-desoxy-6-desmethyltetracycline (minocycline), 6-(2-ethoxy-1-naphthamido)-penicillin (nafcillin), 1-ethyl-1,4-dihydro-7-methyl-4-oxo-1,8-naphthyridin-3-carboxylic acid (nalidixic acid), neomycin, 1-ethyl-6-fluoro-1,4-dihydro-4-oxo-7-(1-piperazinyl)-3-quinolinecarboxylic acid (norfloxacin), (±)-9-fluoro-2,3-dihydro-3-methyl-10-(4-methyl-1-piperazinyl)-7-oxo-7H-pyrido[1,2,3-de][1,4]benzoxazin-6-carboxylic acid (ofloxacin), 6-(5-methyl-3-phenyl-4-isoxazolcarboxamido)penicillanic acid (oxacillin), 6-phenoxyacetylamino-penicillanic acid (phenoxymethylpenicillin) and 4-dimethylamino-octahydro-pentahydroxy-1,11-dioxo-6-methyl-naphtacene-2-carbamide (tetracyclin).

Preferred antibiotics are doxycyclin, minocyclin and neomycin
antiseptics such as e.g. alkylbenzyldimethylammonium chloride (benzalkonium chloride), N-benzyl-N,N-dimethyl-2-{2-[p-(1,1,3,3,-tetramethylbuthyl)-phenoxy]-ethoxy}-ethylammonium hydroxide (benzethonium chloride), cetyltrimethylammonium hydroxide (cetrimonium bromide), 1,1′-hexamethylen-bis-[5-(p-chlorophenyl)-biguanide] (chlorohexidine), N¹,N¹-decamethylen-bis-(4-aminoquinaldinium hydroxide) (dequalinium chloride), N-(4-chlorophenyl)-N′-(3,4-dichlorophenyl)urea (triclocarbane) and 5-chloro-2-(2,4-dichlorophenoxy)phenol (triclosan).

Preferred antiseptics are e.g. 1,1′-hexamethylene-bis-[5-(p-chlorophenyl)-biguanide] (chlorohexidine).
corticosteroids and their physiologically acceptable salts, such as e.g. 9α-chloro-16β-methylprednisolone (beclomethasone), 9-fluoro-11β,17,21-trihydroxy-16β-methyl-1,4-pregnadien-3,20-dione (betamethasone), 21-chloro-9-fluoro-11β,17-dihydroxy-16β-methyl-1,4-pregnadien-3,20-dione (clobetasole), 17,21-dihydroxy-pregn-4-en-3,11,20-trione (cortisone), 11β,16α,17α,21-tetrahydroxy-1,4-pregnadien-3,20-dione-16,17-acetone acetal (desonide), 9-fluoro-11β-17,21-trihydroxy-16α-methylpregna-1,4-dien-3,20-dione (dexamethasone), 9α,11β-dichloro-6α-fluoro-21-hydroxy-16α,17α-(isopropylidenedioxy)-pregna-1,4-dien-3,20-dione (flucloronide), 6α,9α-difluoro-16α,17α-isopropylidenedioxy-corticosterone (fluocinolonacetonide), 6α,9α-difluoro-16α,17α-isopropylidenedioxy-corticosterone-acetate (fluocinonide),6α-fluoro-11β,21-dihydroxy-16α,17-isopropylidenedioxy-4-pregnen-3,20-dione (fludroxycortide), 3-(2-chloroethoxy)-9α-fluoro-6-formyl-11β,21-dihydroxy-16α,17α-isopropylidenedioxypregna-3,5-dien-20-one (formocortal), 21-chloro-9α-fluoro-11β-hydroxy-16α,17α-isopropylidenedioxy-4-pregnen-3,20-dione (halcinonide),17α-hydroxycorticosterone (hydrocortisone), 11β,17,21-trihydroxy-6α-methyl-1,4-pregnadien-3,20-dione (methylprednisolone), 11β,17,21-trihydroxy-pregna-1,4-dien-3,20-dione (prednisolone), 17α,21-dihydroxypregna-1,4-dien-3,11,20-trione (prednisone), 9-fluoro-16α-hydroxyprednisolone (triamcinolone) and triamcinolone-16α,17-acetonide (triamcinolone acetonide).

Preferred corticosteroids are 11β,16α,17α,21-tetrahydroxy-1,4-pregnadien-3,20-dione-16,17-acetone acetal (desonide), 9α,11β-dichloro-6α-fluoro-21-hydroxy-16α,17α-(isopropylidenedioxy)-pregna-1,4-dien-3,20-dione (flucloronide), 6α,9α-difluoro-16α,17α-isopropylidenedioxy-corticosterone (fluocinolonacetonide), 6α,9α-difluoro-16α,17α-isopropylidenedioxy-corticosterone-acetate (fluocinonide), 6α-fluoro-11β,21-dihydroxy-16α,17-isopropylidenedioxy-4pregnen-3,20-dione (fludroxycortide), 3-(2-chloroethoxy)-9α-fluoro-6-formyl-11β,21-dihydroxy-16α,17α-isopropylidenedioxypregna-3,5-dien-20-one (formocortal), 21-chloro-9α-fluoro-11β-hydroxy-16α,17α-isopropylidenedioxy-4-pregnen-3,20-dione (halcinonide), triamcinolone-16α,17α-acetonide (triamcinolone acetonide).

Specific examples of combinations of active substances are:
combinations of corticosteroids with antimycotics, antibiotics or antiseptics. A preferred combination is e.g. (±)-cis-1-acetyl-4-{4-([2-(2,4-dichlorophenyl)-2-(1H-imidazole-1-ylmethyl)-1,3-dioxolane-4-yl]methoxy)phenyl}piperazine (ketoconazole) and 11β,16α,17α,21-tetrahydroxy-1,4-pregnadien-3,20-dione-16,17-acetone acetal (desonide).
combinations of antimycotics of synthetic origin with antimycotics of natural origin. A preferred combination is bis-phenyl-(2-chloro-phenyl)-1-imidazolylmethane (clotrimazole) with tea tree oil.
combinations of various antimycotics of natural origin. A preferred combination is lavender oil, tea tree oil and Australian blue cypress oil.

Suitable nurturing active ingredients according to the invention are above all vital nutrients and anabolic substances preferably selected from the group of amino acids, vitamins and minerals.

Preferred amino acids are (S)-2,6-diaminohexane acid (lysine), ®-2 amino-3-mercaptopropionic acid (cysteine) and especially 2-pyrrolidinecarbonic acid (L-proline). With L-proline an anabolic substance was found which proved suitable in the nail care and repair. As yet L-proline has only been mentioned as a facultative component in cosmetic products for nail care containing either sulfurised amino acids or a derivative thereof as active component (EP-A-0 534 810).

Preferred vitamins are cis-2-(4-carboxybutyl)-3,4-ureidotetrahydrothiophene (biotin), (±)-2,4-dihydroxy-N-(3-hydroxypropyl)-3,3-dimethylbutyramide (panthenole), D(+)-2,4-dihydroxy-N-(3-hydroxypropyl)-3,3-dimethylbutyramide (dexpanthenole).

Preferred minerals are inorganic and organic calcium-, magnesium- and zinc compounds, particularly as organic salts such as glycerophosphate or lactate.

Specific combinations of vital nutrients and anabolic substances are:
combinations of 2-pyrrolidine carboxylic acid (L-proline) with one or more further nutrient and anabolic substances selected from the group of the amino acids, the vitamins and the mineral substances. Preferred combinations of 2-pyrrolidine carboxylic acid (L-proline) with one or more nutrient and anabolic substances are combinations with (S)-2,6-diaminohexanoic acid (lysine), (R)-2-amino-3-mercaptopropionic acid (cystein), gelatine, cis-2-(4-carboxybutyl)-3,4-ureidotetrahydrothiophene (biotin), (±)-2,4-dihydroxy-N-(3-hydroxypropyl)-3,3-dimethylbutyric acid (panthenol), D(+)-2,4-dihydroxy-N-(3-hydroxypropyl)-3,3-dimethylbutyric acid (dexpanthenol) and inorganic or organic calcium, magnesium or zinc compounds.

The topical application products according to the invention can in addition to one or more active substances and one or more compound of the formula I, contain physiologically compatible adjuvants. Suitable adjuvants of this kind are e.g. terpenes or terpene containing oils, alcohols, ketones, fatty acid esters, polyglycols, tensides, urea, antioxidants and complexing agents.

Suitable terpenes are acyclic, monocyclic and bicyclic terpenes as well as oils containing these terpenes. Examples of acyclic terpenes are acyclic terpene hydrocarbons, such as e.g. myrcene, acyclic terpene alcohols, such as e.g.citronellol and geraniol, as well as acyclic terpene aldehydes and ketones, such as e.g. citral, α-ionone and β-ionone. Examples of monocyclic terpenes are monocyclic terpene hydrocarbons, such as e.g. α-terpinene, γ-terpinene and limonene, monocyclic terpene alcohols such as e.g. thymol, menthol, cineol and carvacrol as well as monocyclic terpene ketones such as e.g. menthone and carvone. Examples of bicyclic terpenes are terpenes from the carane group such as e.g. carone, terpenes from the pinane group, such as e.g. α-pinene and β-pinene as well as terpenes from the bornane group such as e.g. campher and borneol. Particularly suitable terpenes are monocyclic terpene alcohols such as e.g. thymol and menthol. Examples for suitable oils containing terpenes are peppermint oil, cardamom oil, geranium oil, rose oil, thuja oil and thyme oil. Particularly suitable oils are peppermint oil, lavender oil and thyme oil.

Suitable alcohols are branched or unbranched alcohols with 1 to 3 hydroxy groups and 2 to 6 carbon atoms, the hydroxy groups optionally being partly or completely etherified or esterified. Particularly suitable alcohols are ethanol, 1-propanol, 2-propanol (isopropanol), 1,2-propanediol (propylene glycol), 2-phenylethanol (phenylethyl alcohol), 1-butanol (butyl alcohol), ethyleneglycol monomethylether (methoxy ethanol), ethylene glycol monophenylether (phenoxyethanol), 1,2,3-trihydroxypropane (glycerin), ethylacetate, butylacetate, glycerin diacetate (diacetin) and glycerin triacetate (triacetin).

As suitable ketones e.g. acetone and methylethyl ketone (2-butanone) are considered.

As fatty acid esters, esters of saturated or unsaturated, branched or unbranched fatty acids with 8 to 21 carbon atoms are suitable, the alcohol component comprising branched and unbranched alcohols with 1 to 6 carbon atoms. Particularly suitable fatty acid esters are tridecane carboxylic acid isopropylester, tetradecane carboxylic acid isopropyl ester (isopropylmyristate), pentadecane carboxylic acid methylester and 9-octadecenoic acid glycerin monoester (glycerin monooleate).

A suitable polyglycol is e.g. polyglycol 400.

Suitable tensides are e.g. non-ionogenic surface active substances. Particularly suitable tensides are partial fatty acid esters of sorbitan (span), partial fatty acid esters of polyoxyethylene sorbitan (tween), fatty acid esters of polyoxyethylene (myrj) and fatty alcohol ethers of polyoxyethylene (brij).

Suitable antioxidants are e.g. butylhydroxytoluene (BHT), butyl-4-methoxyphenol (BHA), tocopherols and ascorbates.

As complexing agents e.g. ethylene diamine tetraacetic acid (EDTA) and disodium-ethylene diamine tetraacetic acid (Na₂-ETDA) are suitable.

As topical application products according to the invention e.g. solutions, tinctures, emulsions, gels, salves, creams and pastes come into consideration. Preferred topical application forms are solutions. For the development of solutions some active ingredients such as proline need traces of water together with a solution mediator as stabilizer (prevention of discolouration). Suitable solution mediators are low potency alcanols such as methanol, ethanol, propanol and isopropanol as well as acetone.

The invention further concerns a process for the manufacture of the topical application products of the invention, which is characterized in that the individual components are homogenously mixed and optionally heated (up to a maximum of 80° C.) and stirred until a homogenous solution is obtained. The solution obtained is preferably used directly as such for topical application. However, the solution can also be converted into another topical application form by the addition of further physiologically acceptable formulation adjuvants with the aid of conventional solution, mixing and suspension procedures.

Preferably, the topical application products according to the invention are used in solution form. Preferred topical application products according to the present invention contain

| | |
|---|---|
| 0.1 to 20% by weight | one ore more active substances, |
| 1 to 99.90% by weight | one or more compounds of the formula I and |
| 0 to 98.90% by weight | one or more physiologically compatible |
| | adjuvants. |

The invention moreover concerns the use of the topical application products according to the invention for treatment, prevention, after-treatment and supporting treatment of nail diseases and periungual diseases as well as for nail care. Furthermore, the present invention concerns the use of the products of the invention for the treatment of mycotic infections of the hooves, paws and claws of pets and domestic animals.

Topical application products containing antimycotics are e.g. suitable for the following indications:
treatment, prevention and after-treatment of onychomycoses, caused by dermatophytes, yeasts or fungi or mixed infections
treatment, prevention and after-treatment of nail-fungus infections in patients with psoriasis, diabetes or AIDS
supporting treatment of periungual nail infections such as e.g. Candida paronychium.

Topical application products containing antibiotics are suitable e.g. for the following indications:
support of the treatment and/or prevention of nail and periungual infections caused by bacteria.

Topical application products containing antiseptics are suitable e.g. for the following indications:
treatment and prevention of nail and periungual infections caused by unspecific or not identified pathogens.

Topical application products containing corticosteroids or combinations of corticosteroids with antimycotics, antibiotics or antiseptics are suitable e.g. for the following indications:
treatment, prevention, after-treatment or supporting treatment of nail psoriasis or other inflamatory nail and periungual conditions

The pharmaceutic topical application products according to the invention are suitable for the treatment of nail diseases and periungual diseases on toenails and fingernails, as well as for the treatment of diseases of the hooves, paws and claws of pets and domestic animals. The frequency of application of the pharmaceutical products depends on the degree and the localization of the disease. In general, application once to three times a day is sufficient. The solution is then directly applied onto the diseased nail or to the hoof, paw or claw and if required, on the surrounding skin areas concerned. The therapy should be continued for about another two to four weeks after laboratory test show no more traces of fungi, spores or other pathogens, in order to prevent a relapse.

The cosmetic topical application products according to the invention containing one or more nutrient and anabolic substances are suitable for nail care such as e.g. in nail atrophies on toenails and fingernails. Nail atrophies include e.g. fragile, brittle and thin nails as well as dotted or streaky white spots. The preparation is applied upon the cosmetically unsightly nail(s) and if required also on the surrounding skin area. The frequency of application of the preparation depends on the degree and the localization of the atrophy. In general, application once or twice a day is sufficient.

The topical application products of the invention have the advantage that they penetrate the diseased nail together with the active substance within a few days and display their action in the nail bed and the nail root. Through the more rapid onset of the effect and the better penetration, the treatment of nail diseases is as a rule terminated after about two to four months. In this way patient-compliance is clearly improved, since the long duration of treatment required in other methods of treatment is substantially shortened. With diseased skin, in particular periungual skin areas, the healing process and the nurturing effect set in faster, since the active substance penetrates sufficiently and rapidly into the skin. The nail care should as a rule be carried out for one month. For maintenance of the healthy nail substance the nail care substance can also be used over a longer period of time.

The present invention can be visualized by the following examples:

### EXAMPLE 1

### Clotrimazole Solution 1%

| | | |
|---|---|---|
| Clotrimazole | 1.0 | g |
| 1-Hexanol | ad 100.0 | ml |

Clotrimazole is dissolved in 100 ml 1-hexanol under stirring.

### EXAMPLE 2

### Amorolfine Solution 1%

| | |
|---|---|
| Amorolfine | 1.0 g |
| Ethanol | 1.0 g |
| 1-Hexanol | 60.0 g |
| 1-Heptanol | 28.0 g |

Amorolfine is stirred into the ethanol, 1-hexanol and 1-heptanol mixture until a homogenous solution is obtained.

### EXAMPLE 3

### Australian Blue Cypress Oil, Tea Tree Oil and Lavender Oil Solution 6%

| | |
|---|---|
| Australian blue cypress oil | 2.0 g |
| Tea tree oil | 2.0 g |
| Lavender oil | 2.0 g |
| Isoamylacetate | 94.0 g |

The mixture is stirred until a homogenous solution is obtained.

### EXAMPLE 4

### Proline Solution 2.0%

| | |
|---|---|
| L-Proline | 2.0 g |
| Water (deionised) up to | 2.0 g |
| Ethanol | 48.0 g |
| Amylacetate | 48.0 g |

L-Proline is dissolved in ethanol and traces of water under stirring. Subsequently amylacetate is added and stirred until a homogenous solution is obtained.

### EXAMPLE 5

### Amorolfine Solution 1%

| | |
|---|---|
| Amorolfine | 1.0 g |
| Ethanol | 10.0 g |
| 1-Hexylacetate | 89.0 g |

Amorolfine is stirred into the ethanol and hexylacetate mixture until a homogenous solution is obtained.

### EXAMPLE 6

### Terbinafine Solution 1%

| | |
|---|---|
| Terbinafine Base | 1.0 g |
| Isoamylacetate | 99.0 g |

The substances are weighted out into a beaker and stirred until a homogenous solution is obtained.

### EXAMPLE 7

### Cream with Amylacetate

| | |
|---|---|
| H₂O free of ions | 720 g |
| Carbopol Ultrez 10 | 9.6 g |
| Glycerine | 24.0 g |
| Sunflower oil | 216.0 g |
| Emulgade 1000 NI | 45.6 g |
| Lanette N | 9.6 g |
| Amylester | 36.0 g |
| Phenonip | 9.6 g |
| Triethanolamine | |

Under stirring, disperse carbopol in H₂O and let it mascerate. Add glycerine and heat up to +50° C.

Under stirring and heating up to 70° C., make a clear solution out of sunflower oil, emulgade and lanette.

Amylester is added to the fatphase and is homogenized with the waterphase under strong stirring. Phenopip is blended in.

Cool down the cream and adjust the pH-value to 5.5 with triethanolamine.

1-15. (canceled)

16. A topical application product for treatment of nail diseases and nail care comprising
a) one or more therapeutic or nurturing active substances,
b) one or more compounds of formula I
R—O—R₁ (I)
where
R represents a straight or branched alkyl residue with 5-8 carbon atoms and
R₁ represents a formyl group or an acetyl group, and
c) if necessary a physiologically compatible adjuvant.

17. A topical application product according to claim 16, wherein R is a C₅-C₇ alkyl residue.

18. A topical application product according to claim 16, wherein R a C₆-alkyl residue.

19. A topical application product according to one of claims 16-18, wherein the alkyl residue is 1-hexanol.

20. A topical application product according to claim 16 or 17, comprising a mixture of two or more formates or acetates of C₅-C₇ alkanols as compounds of formula I.

21. A topical application product according to claim 16, comprising one or more C₅-C₆ alkyl acetates as a compound of formula I.

22. A topical application product according to claim 16, wherein the active substance is one or more active substances selected from the group of synthetic or natural antimycotics, antibiotics or antiseptics and corticosteroids.

23. A topical application product according to claim 16, wherein the active substance is one or more nurturing and curing components selected from the group of nutrient and anabolic substances.

24. A topical application product according to claim 16, further comprising one or more adjuvants selected from the group of terpenes or terpene containing oils, alcohols, ketones, fatty acid esters, polyglycols, tensides, urea, antioxidants and complexing agents, contained in gels, salves, creams and pastes.

25. A topical application product according to claim 16, comprising 0.1 to 20 percent by weight of one or more active substances, 1 to 99.90 percent by weight of one or more compounds of formula I, and 0 to 98.90 percent by weight of adjuvants.

26. A method of treating, preventing, after-treating or support-treating nail diseases and periungual diseases which comprises administering a product according to claim 16 to a patient in need thereof.

27. A method of caring for nails which comprises administering a product according to claim 16 to the nails.

28. A method of treating mycotic infections of the hooves, paws and claws of pets and domestic animals which comprises administering a product according to claim 16 to a pet or domestic animal in need thereof.
